(19) United States
(12) Patent Application Publication
(10) Pub. No.: US 2025/0073304 A1
(43) Pub. Date: Mar. 6, 2025

(54) DRUGS, EXPRESSION VECTORS, AND THEIR APPLICATIONS IN THE TREATMENT OF NAFLD AND RELATED DISEASES

(71) Applicant: Wuhan University, Wuhan (CN)

(72) Inventors: Feng LI, Wuhan (CN); Dong MEN, Wuhan (CN); Yan LIN, Wuhan (CN); Mingkun YANG, Wuhan (CN); Jie XIONG, Wuhan (CN); Li HUANG, Wuhan (CN)

(73) Assignee: Wuhan University, Wuhan (CN)

(21) Appl. No.: 18/818,491

(22) Filed: Aug. 28, 2024

(30) Foreign Application Priority Data
Aug. 29, 2023 (CN) 2023110934878

(51) Int. Cl. A61K 38/16; A61P 1/16; C12N 15/85

(52) U.S. Cl. CPC A61K 38/164; A61P 1/16; C12N 15/85; C12N 2750/14143

(57) ABSTRACT

The application provides drugs, expression vectors, and their applications in the treatment of NAFLD and related diseases, pertaining to the technical field of targeted drugs. The drugs include streptavidin or a recombinant expression vector for streptavidin. Streptavidin can bind to acetyl CoA carboxylase 1, alter its subcellular localization within mammalian cells, and ultimately inhibit de novo lipogenesis. Ectopic expression of streptavidin can inhibit lipid accumulation within mammalian cells. Compared with chemical small-molecule drugs, streptavidin, as a protein drug, exhibits relatively lower liver and kidney toxicity. Streptavidin might be a relatively mild inhibitor of acetyl CoA carboxylase, avoiding a severe side effect of hyperlipidemia. Furthermore, streptavidin is very stable, which might be conducive to drug delivery and adaptable to different administration methods.

### CROSS-REFERENCE TO RELATED APPLICATIONS

The application claims priority to Chinese patent application No. CN202311093487.8, filed on Aug. 29, 2023, the entire contents of which are incorporated herein by reference.

### SEQUENCE LISTING

The sequence listing xml file submitted herewith, named “DRU.xml”, created on Aug. 20, 2024, and having a file size of 3,900 bytes, is incorporated by reference herein.

### TECHNICAL FIELD

The present application relates to the technical field of targeted drugs, and in particular to drugs, expression vectors, and their applications in the treatment of NAFLD and related diseases.

### BACKGROUND

Non-alcoholic fatty liver disease (NAFLD) is a potentially serious liver disease that affects approximately one-quarter of the global adult population, causing a substantial burden of ill health with wide-ranging social and economic implications. However, there are few drugs approved for the clinical treatment of NAFLD. Elevated hepatic de novo lipogenesis (DNL) is a prominent feature of NAFLD. Previous studies have clearly shown that inhibition of acetyl-CoA carboxylases (ACCs), which play a pivotal role in the hepatic DNL pathway, can effectively inhibit hepatic steatosis and fibrosis, indicating that ACC are an attractive therapeutic target for NAFLD or non-alcoholic steatohepatitis (NASH). Unfortunately, abrupt inhibition of ACC by chemical inhibitors can cause a cellular compensatory reaction by the activation of SREBPI and thus lead to hyperlipidemia, a confirmed side effect that limits the clinical application of ACC inhibitors. Therefore, there is an urgent need to develop a novel ACC-targeting strategy.

Streptavidin (SA) is a protein isolated from the bacterium Streptomyces streptavidinii and is well known for its extraordinarily high affinity for biotin. Streptavidin is used extensively in molecular biology and bionanotechnology. To date, the biochemical properties of SA in mammalian cells and the potential role of SA in the treatment of NAFLD has never been investigated.

### SUMMARY

The role of streptavidin in the treatment of NAFLD and related diseases has not been documented or researched in the prior art. In order to solve this problem, the present application explores the new use of streptavidin as an inhibitor of acetyl CoA carboxylase for the preparation of drugs aimed at treating NAFLD and related diseases. This is achieved by studying the relationship between streptavidin derived from bacteria and these conditions.

To achieve the above purpose, the embodiments of the application discloses at least the following technical solutions:

Firstly, the embodiments disclose a method for constructing a recombinant expression vectors for streptavidin, wherein the amino acid sequence of streptavidin is shown in SEQ ID NO:1, and the construction method comprises:
PCR amplification of streptavidin gene,
obtaining the target gene fragment for the expression of streptavidin, and
connecting the target gene fragment with the expression vector to obtain a recombinant expression vector for streptavidin.

Furthermore, the primers used for PCR amplification of streptavidin gene are:
forward primer, the nucleotide sequence of which is as shown in SEQ ID NO:2;
reverse primer, the nucleotide sequence of which is as shown in SEQ ID NO:3.

Secondly, the embodiment discloses the recombinant expression vector for streptavidin obtained by the said construction method.

Thirdly, the embodiments disclose a drug for the treatment of NAFLD and related diseases, comprising streptavidin, the amino acid sequence of which is as shown in SEQ ID NO: 1, and the streptavidin can bind to acetyl COA carboxylase 1 (ACC1) and alter its subcellular localization after implantation into a mammalian body.

Furthermore, the streptavidin in the drug is obtained through the said recombinant expression vector for streptavidin, and the specific steps involved are as follows:
introduction of the recombinant expression vector for streptavidin into host cells;
screening for high-expression positive host cells, cultivation of these cells and induced expression.

Fourthly, the embodiments disclose another drug for the treatment of NAFLD and related diseases, which comprises the said recombinant expression vector for streptavidin. The recombinant expression vector for streptavidin expresses streptavidin in mammalian cells, and upon implantation into a mammalian body, the streptavidin can bind to ACC1 and alter its subcellular localization.

Fifthly, the embodiments disclose the application of the streptavidin and/or the recombinant expression vector for streptavidin in the preparation of drugs for the treatment of NAFLD, NASH and liver cancer.

The present application provides drugs, expression vectors, and their applications in the treatment of NAFLD and related diseases. Compared with the prior art, the present application has at least the following beneficial effects:
1. Compared with chemical small-molecule drugs, streptavidin, as a protein drug provided in this application, has relatively lower liver and kidney toxicity;
2. Streptavidin provided in this application might be a relatively mild inhibitor of acetyl CoA carboxylase, which will not cause the severe side effect of hyperlipidemia;
3. Streptavidin provided in this application is very stable, which might be conducive to drug delivery and adaptable to different administration methods.

### BRIEF DESCRIPTION OF DRAWINGS

FIG. 1 shows the subcellular localization of ACC1 provided in an embodiment of the present application.

FIG. 2 shows the electropherogram of SA combined with ACC1 provided in an embodiment of the present application.

FIG. 3 shows the results of Oil Red O staining provided in an embodiment of the present application.

FIG. 4 shows the pathological results of mouse livers provided in an embodiment of the present application.

FIG. 5 shows the results of isotope tracing experiment provided in an embodiment of the present application.

### DETAILED DESCRIPTION OF THE EMBODIMENTS

In order to clarify and make the purpose, technical solution, and advantages of the present application more comprehensible, the present application will be further elaborated with reference to the following embodiments. It should be understood that the specific embodiments described herein are merely used to explain the present application, and are not used to limit the present application. The reagents not specifically described in the present application are conventional reagents that can be obtained from commercial sources, and the methods not explicitly described are conventional experimental methods that can be learned from the prior art.

It should be noted that the terms “first”, “second”, and similar expressions used in the present application's specification, claims, and the aforementioned figures are solely intended to distinguish between similar objects and do not necessarily indicate a specific order or sequence. Furthermore, they are not intended to limit the subsequent technical features in any substantial manner. It should be understood that, in appropriate cases, such data can be interchanged, allowing the embodiments of the present application described herein to be implemented in an order different from the one illustrated or described. Additionally, the terms “including” and “having”, along with their variations, are meant to encompass non-exclusive inclusion. For instance, a process, method, system, product, or device that includes a series of steps or units is not limited solely to those steps or units explicitly listed but may also include other steps or units inherent to the process, method, product, or device, even if they are not explicitly mentioned.

In order to better understand the present application, rather than limiting the scope of the present application, all numerical values, such as quantities and percentages, and other numerical values used in this application, can be modified by the term “approximately” in all cases. Therefore, unless otherwise specified, the numerical parameters listed in the specification and the claims are approximate values that may be changed depending on the different ideal properties being sought. Each numerical parameter should at least be considered as being obtained by conventional rounding methods based on the reported significant figures.

Based on this, the embodiments provide a method for constructing a recombinant expression vector for streptavidin, wherein the amino acid sequence of streptavidin is shown in SEQ ID NO:1, and the construction method comprises: PCR amplification of streptavidin gene, obtaining the target gene fragment for the expression of streptavidin, and connecting the target gene fragment with the expression vector to obtain a recombinant expression vector for streptavidin.

In some embodiments, the primers used for PCR amplification of streptavidin gene are: forward primer, the nucleotide sequence of which is as shown in SEQ ID NO:2; reverse primer, the nucleotide sequence of which is as shown in SEQ ID NO:3.

Based on this, the embodiments provide recombinant expression vectors for streptavidin obtained by the said construction methods.

Based on this, the embodiments provide a drug for the treatment of NAFLD and related diseases, comprising streptavidin (SA), the amino acid sequence of which is as shown in SEQ ID NO:1, and the streptavidin can bind to ACC1 and alter its subcellular localization after implantation into a mammalian body. The alteration of subcellular localization will render ACC1 unable to exert its normal enzyme activity, thereby inhibiting de novo lipogenesis (DNL). Consequently, SA can be considered as a potential drug for the treatment of NAFLD and related diseases.

In some embodiments, the streptavidin in the drug is obtained through the said recombinant expression vector for streptavidin, and the specific steps involved are: introduction of the recombinant expression vector for streptavidin into host cells; screening for high-expression positive host cells, cultivation of these cells and induced expression.

Based on this, the embodiments also provide another drug for the treatment of NAFLD and related diseases, which comprises the said recombinant expression vector for streptavidin. The recombinant expression vector for streptavidin expresses streptavidin in mammalian cells, and upon implantation into a mammalian body, the streptavidin can bind to ACC1 and alter its subcellular localization.

Based on this, the embodiments disclose an application of the streptavidin and/or the recombinant expression vector for streptavidin in the preparation of drugs for the treatment of NAFLD, NASH and liver cancer.

In some embodiments, streptavidin can significantly alleviate NASH in mice. To investigate this, an animal model of NASH was established by feeding mice a high fat, high cholesterol (HFHC) diet for 7 weeks. Subsequently, an adeno-associated virus (AAV) vector was utilized to specifically deliver cDNA encoding SA into the livers of mice that had been fed a HFHC diet for an additional 9 weeks of treatment. After 9 weeks of treatment, the mice were sacrificed, and their livers were subjected to physiological and pathological analyses. The results indicated that the SA treatment group showed a notably reduced accumulation of liver lipids and a decreased severity of liver fibrosis, without significant elevation in liver lipid levels. Additionally, the SA treatment, through its effect on the expression of streptavidin, significantly mitigated the onset and progression of NASH in mice, without inducing notable side effects.

In some embodiments, streptavidin can inhibit the accumulation of intracellular lipids. To investigate this, we utilized two experimental subjects: a human hepatocellular carcinoma cell line, Huh7, and a Huh7 cell line that over-expresses streptavidin. A fatty liver cell model induced by a combination of palmitic acid (PA) and oleic acid (OA) was employed to investigate the function of streptavidin within mammalian cells. It was found that when streptavidin is expressed, the lipid accumulation in Huh7 cells induced by PA+OA stimulation is significantly reduced, indicating that the ectopic expression of streptavidin can inhibit lipid accumulation within mammalian cells.

In some embodiments, streptavidin can significantly inhibit DNL, as demonstrated by the following experiment: Mice that had been treated with streptavidin for 9 weeks were selected as experimental subjects. Their newly synthesized fatty acids were then labeled for 24 hours by gavaging the mice with ¹³C-labeled sodium acetate. After 24 hours, the mice were sacrificed, and their livers were harvested for analysis of the rate of DNL using Ultra-Performance Liquid Chromatography-Mass Spectrometry (UPLC-MS). The experimental results showed that streptavidin significantly inhibited the rate of DNL in the mouse liver. Furthermore, it was found that streptavidin significantly binds to ACC1, suggesting that streptavidin can inhibit DNL by targeting ACC1.

Herein, a more detailed description of the present application is provided in conjunction with specific embodiments. It is emphasized that these embodiments should not be construed as constituting limitations to the scope of the present application.

### 1. Cell Culture

Huh7 cells (human liver cancer cells, purchased from Wuhan Shangen Biotechnology Co., Ltd.) were cultured in High Glucose Dulbecco's modified Eagle's medium (HG-DMEM) supplemented with 10% FBS, and maintained in a constant temperature incubator at 37° C. with 5% CO₂. The cells used in the experiment were not more than 3 months of age, and mycoplasma detection was conducted every 3 months. Cell cryopreservation was performed using complete medium containing 10% DMSO.

### 2. Stable Cell Lines Construction Via Lentivirus Infection

(1) Recombinant Plasmids (pHAGE-3xflag-SA) Construction

SA genes were amplified by PCR with forword and reverse primers (identified as SEQ ID NO:2-3). We used agarose gel electrophoresis to assess the PCR products and excised the desired band, and recovered the SA fragments by DNA Recovery Kit. The SA fragments and flag-tagged vectors (pHAGE) were digested with two restriction enzymes simultaneously, and seperated by agarose gel electrophoresis. We linked the vectors and the SA fragments by using Ligation High Ligase at 16° C. for 2 hours. The constructed plasmids were transformed into DH5α (E. Coli) and cultured at 37° C. overnight. Monoclonal colonies were randomly selected and identified by PCR, and the results of the positive colonies were further confirmed by sequencing analysis. We cultured the colonies with correct sequence and extracted the constructed plasmids for the preparation of the following steps.

### (2) Lentivirus Packaging

HEK293T cells (human embryonic kidney cells 293) were seeded into 6-well plates. When the cell density reached approximately 70% the next day, the cell transfection was conducted. 200 μL serum-free opti-MEM™ medium was added to a 1.5 mL EP tube. 2 μg plasmids (with a mass ratio of pMD2. G: psPAX2: pHAGE-3xlag SA=1:1:2) and 2 μL transfeciton reagents were added to each well, and incubated at room temperature for 20 min when mixed fully. We added 200 μL mixture to the cell medium, gently mixed it, and cultured the cells in an incubator. The lentiviral particles were collected at 48 h and 72 h post-transfeciton and filtered through a 0.4 μm filter, and then kept at −80° C. after being packed separately as virus suspension.

### (3) Cell Transfection Via Lentivirus

The cells were seeded into 6-well plates and transfected when the cell density reached 30-50% the next day. The medium of the cells was removed and replaced with fresh serum-free opti-MEM™. Virus suspension (500 μL) and polybrene were added to each well, in which the final concentration of polybrene was 8 mg/mL. After being mixed, they were further incubated. After 24 hours of infection, we added new virus suspension to the medium, allowing the cells to be transfected for another 24 hours. Finally, the cells were transferred to a selective medium containing purines to screen for positive transformants.

### 3. Immunofluorescence Staining

Coverslips were placed into the 6-well plates, and the cells at a density of about 10% were transfected for 48 h. After transfection, the medium was aspirated, and the cells were rinsed three times with PBS. For immunofluorescence staining, the cells were fixed with 4% paraformaldehyde (1 mL/well) for 10 min, with subsequent aspiration of the fixative. The cells were rinsed with PBS three times and permeabilized using 0.2% Triton X-100 for 10 min. After aspirating the permeabilization solution, the cells underwent three additional rinses with PBS. Following a 10-minute blocking step with 5% BSA, the cells were incubated overnight at 4° C. with primary antibodies (ACC1, diluted in a 1% BSA/PBS solution). The supernatant was discarded, and the cells were rinsed three times with 0.1% PBST. Subsequently, the cells were incubated with secondary antibodies for 1 hour at room temperature in the dark, in a solution of 1% BSA. The secondary antibody solution was discarded, and the cells were rinsed three times with 0.1% PBST. Finally, a drop of scaling liquid containing DAPI was added to the slide as a mounting media. The coverslip was sealed with nail polish to prevent dehydration, and fluorescence images were performed on a confocal microscope.

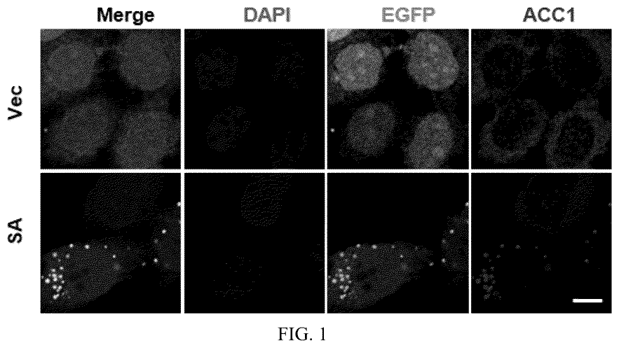

FIG. 1 shows the subcellular localization of ACC1. As shown in FIG. 1, ectopic expression of SA dramatically induces the ACC1 to aggregate together, forming dispersed cytoplasmic bright spots in the cytosol, and these aggregates almost completely colocalize with the SA condensate. In contrast, in the control group, ACC1 was originally located in the cytoplasm, where it was apparently evenly dispersed. Therefore, it is demonstrated that SA can alter the cellular localization of ACC1.

### 4. Immunoprecipitation

HEK293T cells were seeded into 6-well plates, and the next day, the state and density of the cells were observed, with the density being approximately 50%. Subsequently, the indicated expression plasmids were transfected into the respective HEK293T cells using transfection reagents, with approximately the cell amount required for two wells per sample group. After 48 h of transfection, cell samples were harvested (the cell samples were stored at −80° C. and were retrieved when necessary). Each sample was lysed in 600 μL IP lysis buffer for 30 min on ice, and the cell samples were ultrasonicated on ice using a program set at 10% power, with the ultrasound pulse duration of 2 seconds followed by an 8-second pause. The total ultrasonication time was 5 minutes, and the entire process was carried out in the refrigerator. To remove the cell precipitates, the cell lysates were centrifuged at 12,000 rpm for 10 min at 4° C. and the supernatants were collected. For each sample, 15 μL of magnetic beads were mixed and then added to the corresponding supernatant. Tubes containing the magnetic beads were placed in the magnetic separation rack. We then aspirated the residual ethanol from the beads, added 400 μL of 1% TBST, and rinsed the beads three times. 80 μL of cell lysates was prepared as input, and the remaining cell lysates were incubated with indicated magnetic beads and incubated for 1 h at room temperature. Afterwards, the cell lysates were rinsed with 0.1% TBST three times to remove non-specific binding proteins, and the immunocomplex were eluted with 60 μL 2×SDS loading buffer by boiling at 100° C. for 10 min. The magnetic beads were placed on the magnetic rack, and the supernatants were analyzed by Western Blot (WB).

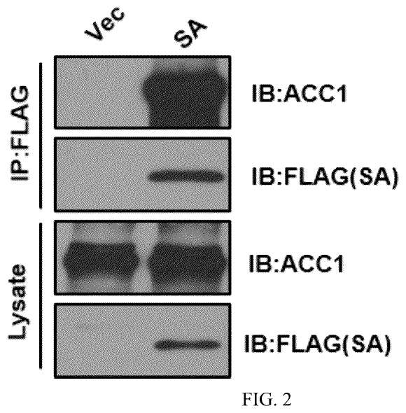

As shown in FIG. 2, the WB results unequivocally demonstrated that streptavidin successfully combined with ACC1, providing clear evidence of their interaction.

5. Oil Red O staining

The cells were cultured in high-fat medium for 24 h and then retrieved from the incubator. Subsequently, the state and density of the cells were observed, with the density being approximately 50%. After aspirating and discarding the culture medium, the cells were thoroughly rinsed three times with PBS to remove any residual medium. Following the rinses, the PBS was carefully aspirated to ensure the cells were as dry as possible. Then we added 4% paraformaldehyde fixative and fix the cells at room temperature for 10 to 15 minutes. After fixing, the cells were rinsed with PBS three times for 3 minutes each, followed by blotting to remove excess PBS. After adding 60% isopropyl alcohol for 30 seconds, we immediately discarded the alcohol and thoroughly rinsed the cells with PBS to remove any remaining isopropyl alcohol. After thoroughly removing excess PBS, the cells were left to dry naturally in a well-ventilated area. Upon drying, the bottom of the culture dish turned white. The Oil Red O Working Solution was prepared by combining Oil Red O stock solution with PBS in a 3:2 ratio by volume. The mixture was then allowed to stand at room temperature for 10 minutes. Subsequently, the solution was filtered through a 0.45 μm filter, rendering it ready for use. After adding the Oil Red O Working Solution to the cells, we observed them under the microscope. When the presence of red lipid droplets became evident, we promptly discarded the Oil Red O Working Solution. After staining, we rinsed the cells with PBS three times to remove excess Oil Red O Working Solution. If a layer of floss formed on the liquid surface, we carefully absorbed it to prevent deposition on the bottom or walls of the dish. Following differentiation, we further rinsed the cells three times with PBS, optionally using 60% isopropyl alcohol during the rinses to enhance the cleaning process. After adding PBS for immersion, it was advised to adopt the Ph2 mode and capture multiple photographs at different magnifications.

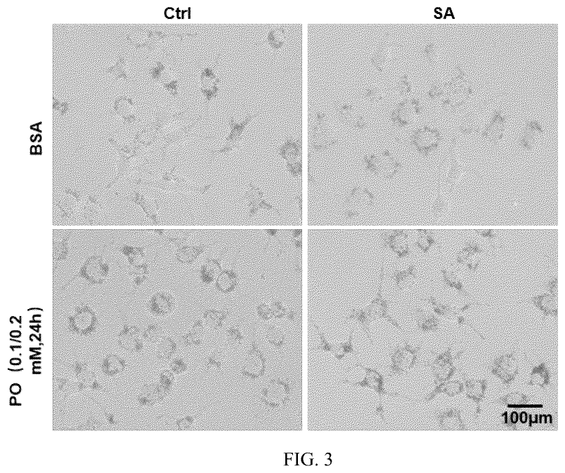

FIG. 3 presents the result of Oil Red O staining, with the BSA treatment group serving as the control. In this setup, PO denoted the application of a PA (palmitic acid) to OA (oleic acid) ratio of 0.1/0.2 mM for 24 h. According to FIG. 3, it becomes evident that the SA treatment group exhibited a notably lower content of lipid droplets compared to the control group. This observation underscores the fact that the ectopic expression of SA effectively diminished the intracellular accumulation of lipids.

### 6. Establishment of NASH Mouse Models

C57BL/6J background male mice (7-8 weeks of age) used for NASH model establishment were purchased from Beijing Vital River Laboratory Animal Technology Co., Ltd. (Beijing, China). To induce NASH, mice were fed an HFHC diet (protein, 14%; fat, 42%; carbohydrates, 44%; cholesterol, 0.2%). After 7 weeks of feeding, the mice were randomly assigned to two groups, whereupon they received, via tail vein injection, Adeno-associated viruses (AAV, purchased from Hanheng Biotechnology Shanghai Co., Ltd) either containing SA cDNA or lacking it. The mice were sacrificed after 16 weeks of being fed the HFHC diet, in order to procure their livers for subsequent experimental procedures. Pathological examinations were conducted by means of Oil Red O staining, H&E staining, and Masson stain.

### 7. Measurements of Hepatic De Novo Lipogenesis (DNL)

### (1) ¹³C-Labled Hepatic DNL

[1, 2-¹³C]-acetate was prepared 24 h in advance and the mice were treated [1, 2-¹³C]-acetate by oral gavage at a dose of 1 g/kg for 24 h. Then mice were sacrificed 24 h later, and their livers tissues were promptly collected, frozen in liquid nitrogen, and stored at −80° C. for later use.

(2) Extraction of Total Fatty Acids from Mouse Livers

50 mg liver tissues was added to 500 μL extraction solvent consisting of a mixture of dichloromethane and methanol in a 2:1 ratio. The tissues were subsequently homogenized on ice for 2 to 3 times, with each time lasting no longer than 10 seconds. Following each homogenization step, water was added in a 1:4 ratio, and the homogenates were thoroughly mixed by vortex for 30 seconds. Let the mixture stand for 5 minutes. And this entire process was repeated three times. Following complete extraction, the homogenates were centrifuged 3000 rpm for 20 min at room temperature. After centrifuging the mixture, we confirmed the absence of any obvious stratification. In response, we re-vortexed the mixture and then centrifuged it further at 12,000 rpm for 20 minutes to achieve stratification. After stratification, an equal volume of the organic phase (the lower layer) was transferred into a new 1.5 mL EP tube. The organic solvent was dried to a thin film form under the protection of nitrogen (during the nitrogen drying process, the airflow should not be too large, and it should not be heated). Then the fatty acid was resuspended in 1 mL methanol/water containing 0.3 M KOH (V:V=90:10, 900 μL methanol mixed with 100 μL 3M KOH), and saponified in water bath at 80° C. for 1 h. After saponification, 0.1 mL formic acid was added for acidification, and the mixture was thoroughly mixed by vortex. ImL of n-hexane was added and the mixture was mixed by vortex and standed for stratification. Subsequently the upper organic phase was taken to a new 1.5 mL EP tube. An additional ImL of n-hexane was added, and the upper organic phase was extracted again. The extracted organic phases were combined, and the combined organic phase was then dried under nitrogen protection (during the nitrogen drying process, the airflow should not be too large, and it should not be heated).

### (3) Fatty Acid Analysis

As for fatty acid analysis, samples were resuspended in 120 mL of dichloromethane (CH₂Cl₂)/Methanol (MeOH) (V:V=1:1). Fatty acid analysis was performed on the UPLC system was coupled to a Q-Exactive HF orbitrap mass spectrometer (Thermo Fisher, CA) equipped with a heated electrospray ionization (HESI) probe. The separation of lipid extracts was performed on a CORTECS C18 (100×2.1 mm, 2.7 mm) column (Waters, USA) using a binary solvent system, with mobile phase A consisting of ACN: H2O (60:40), 10 mM ammonium acetate, and mobile phase B of IPA: ACN (90:10). Samples were eluted by following the 18 min linear gradient with flow rate of 250 mL/min: 0 min, 30% B; 2.5 min, 30% B; 8 min 50% B; 10 min, 98% B; 15 min 98% B; 15.1 min, 30% B; 18 min 30% B. The temperature of the column chamber and sample tray were held at 40° C. and 10° C. respectively. In negative ion mode, full scan is obtained in the mass range of 150-600 m/z with a resolution of 70,000. The source parameters are as follows: spray voltage: 3000 v; capillary temperature: 320° C.; heater temperature: 300° C.; sheath gas flow rate: 35 Arb; auxiliary gas flow rate: 10 Arb. Data analysis was performed by the tracefinder3.2 (Thermo Fisher, CA). Accurate quality was determined according to the C6-C30 internal mass spectrometry database. The mass tolerance for precursor search was set at 10 ppm. Metabolites were assigned based on the exact mass of the precursor ion. For relative quantification, the chromatographic peak area method was employed. Peak alignment permitted a 0.25 min offset for retention time (RT).

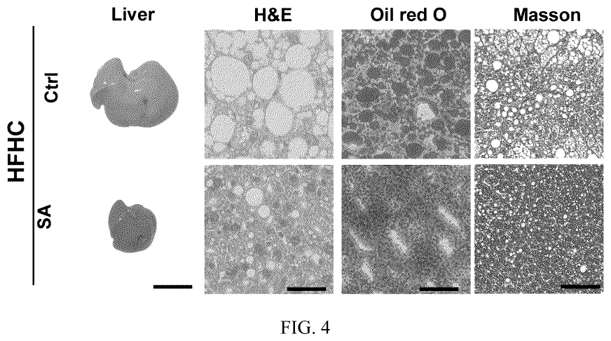

FIG. 4 is the pathological results of the mouse livers. As shown in FIG. 4, the mice in the SA treatment group exhibited a significant alleviation of NASH, evidenced by the restoration of liver volume, decreased lipid accumulation, and reduced fibrosis.

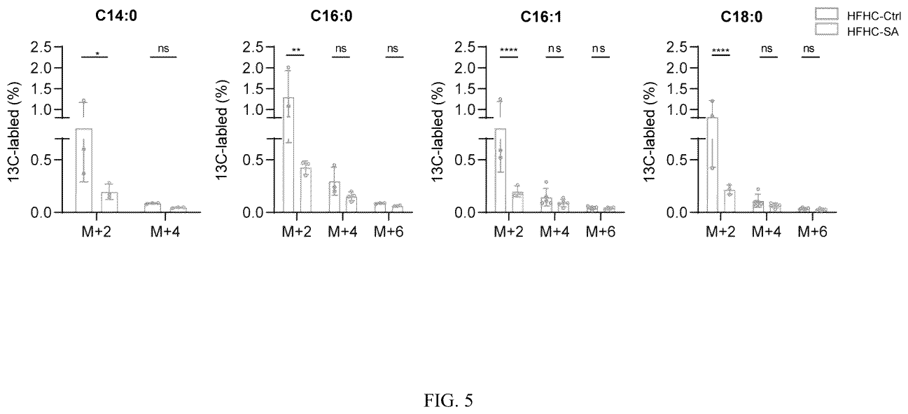

FIG. 5 depicts the results of isotope tracing, wherein the abundance of isotopically labeled fatty acids, specifically C14:0, C16:0, C16:1, and C18:0, in the de novo lipogenesis (DNL) pathway serves as an indicator of DNL rate. As evident in FIG. 5, the proportion of isotopically labeled molecules in the SA treatment group is significantly reduced compared to the control group, indicating a significant inhibition of the DNL rate in mice subjected to SA treatment.

The above is only the preferred specific implementation method of this application, and the scope of this application is not limited to this. Any changes or replacements that can be easily thought of by technical personnel familiar with the technical field within the scope of the disclosure in this application should be covered within the scope of this application.

1. A drug for the treatment of NAFLD comprising streptavidin with the amino acid sequence of SEQ ID NO: 1, said streptavidin binds to acetyl COA carboxylase 1 and alters its subcellular localization after implantation into a mammalian body.

2. A drug according to claim 1, wherein the streptavidin in the drug is obtained through the said recombinant expression vector for streptavidin, and the specific steps involved are as follows:
introduction of the recombinant expression vector for streptavidin into host cells;
screening for high-expression positive host cells, cultivation of these cells and induced expression;
the construction method of the recombinant expression vector comprises:
PCR amplification of streptavidin gene,
obtaining the target gene fragment for the expression of streptavidin, and
connecting the target gene fragment with the expression vector to obtain a recombinant expression vector for streptavidin.

3. A drug for the treatment of NAFLD, comprising a recombinant expression vector for streptavidin, which expresses streptavidin in mammalian cells; the amino acid sequence of the said streptavidin is as shown in SEQ ID NO:1; upon implantation into a mammalian body, the streptavidin can bind to ACC1 and alter its subcellular localization;
the construction method of the recombinant expression vector comprises:
PCR amplification of streptavidin gene,
obtaining the target gene fragment for the expression of streptavidin, and
connecting the target gene fragment with the expression vector to obtain a recombinant expression vector for streptavidin.

4. An application of streptavidin and/or the recombinant expression vector for streptavidin in the preparation of drugs for NAFLD and NASH; the amino acid sequence of the streptavidin is as shown in SEQ ID NO:1;
the construction method of the recombinant expression vector comprises:
PCR amplification of streptavidin gene,
obtaining the target gene fragment for the expression of streptavidin, and
connecting the target gene fragment with the expression vector to obtain a recombinant expression vector for streptavidin.


SEQUENCE LISTING (3 sequences)

SEQ ID NO: 1 - protein, 159 aa, synthetic construct
DPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 2 - DNA, 26 bp, synthetic construct
  source 1..26: mol_type = other DNA
ggggctagcgacccgagcaaagattc

SEQ ID NO: 3 - DNA, 24 bp, synthetic construct
  source 1..24: mol_type = other DNA
gggctcgagttattgttgaactgc